(19) United States
(12) Patent Application Publication
(10) Pub. No.: US 2025/0197788 A1
(43) Pub. Date: Jun. 19, 2025

(54) Bioreactor System for Valorization of Corn Ethanol and Brewery Byproducts

(71) Applicant: Capra Biosciences, Inc., Alexandria, VA (US)

(72) Inventors: Andrew P. Magyar, Acton, MA (US); Elizabeth Onderko, Alexandria, VA (US); Dane Reed, Lees Summit, MO (US); Mark Poole, Raleigh, NC (US)

(21) Appl. No.: 18/849,743

(22) PCT Filed: Mar. 31, 2023
(86) PCT No.: PCT/US2023/017053

§ 371 Date: Sep. 23, 2024

Related U.S. Application Data
(60) Provisional application No. 63/326,428, filed on Apr. 1, 2022.

(51) Int. Cl. C12M 1/00; C12M 1/12; C12P 23/00

(52) U.S. Cl. CPC C12M 23/58; C12M 25/16; C12M 29/18; C12M 43/00; C12P 23/00

(57) ABSTRACT

Described herein are methodologies for employing biofilm bioreactors and hydrocarbonoclastic organisms to upscale the corn ethanol byproduct thin stillage or by products from distilleries or breweries into value-added hydrophobic chemicals like retinoids and lubricants.

### RELATED APPLICATIONS

This application claims the benefit under 35 U.S.C. § 119(e) of U.S. Provisional Application No. 63/326,428, filed on Apr. 1, 2022, which is incorporated herein by reference in its entirety.

### BACKGROUND OF THE INVENTION

Most commercially-available biosynthesized chemicals today are produced using fermentation in a batch process where suspended cells in a large vat of refined sugar-containing growth media concomitantly grow and produce a product that is eventually isolated from the aqueous media or the cells themselves.

In contrast, biofilm bioreactors allow for the use of more diverse and complex carbon sources as well as continuous-flow product synthesis, which can be more efficient than batch style synthesis and extraction cycles. Hydrocarbonoclastic organisms are capable of metabolizing hydrocarbons as their food source. These organisms can possess a high carbon flux pathway useful for building value-added chemicals while also being solvent tolerant, thus enabling continuous flow extraction techniques.

The continuous-flow biofilm bioreactor design (Glaven et al., U.S. Pat. Application Pub. No. US 2021/0253990 A1, hereinafter Glaven, incorporated herein by this reference in its entirety) combines the benefits of biosynthesis with a water-, energy-, and time-saving solvent extraction step to produce complex hydrophobic chemicals from low-value feedstocks in a cost-effective manner.

Dry-mill corn ethanol plants ingest corn and mix that corn with yeast, water, and enzymes to convert the available corn starch into sugar. Ethanol plants then ferment that sugar into their target molecule, ethanol. The fermented liquid passes through a series of distillation columns to extract the ethanol, the remaining byproduct typically being referred to as “whole stillage”. The whole stillage containing both liquid and suspended solids left-over from fermentation then enters a centrifuge for separation. The extracted solids are deemed wet cake and sent to a dryer for dehydration into the animal feed additive called dried distillers grains (DDG)—mostly for the bovine, swine, and poultry industries. Thin stillage, a nutrient-rich liquid composed mostly of water, is the liquid portion exiting the centrifuge.

Ethanol plants still use and profit from thin stillage as an oil source and animal feed additive. Typically, they first dehydrate the thin stillage to enable more efficient processing for both oil extraction and final drying for feed additives. Ethanol Plants pass thin stillage through a series of evaporators to remove water content, forming a concentrated thin stillage called condensed (or concentrated) distillers solubles (also known as syrup) or CDS.

Ethanol Plants then spin the CDS or “syrup” in a large centrifuge to extract the very profitable corn oil currently sold as a distinct byproduct marketed as “distillers corn oil” to biodiesel plants and other customers. The “de-oiled” syrup is then returned for more evaporation before the syrup is added to the wet cake (solids portion of the whole stillage centrifuge detailed above) to increase its nutrient content for animal feed. Wet cake dried without re-adding the syrup is the Dried Distillers Grains (DDG). Wet cake with syrup re-added is called Dried Distillers Grains with Solubles (DDGS). In rare cases, Ethanol Plants sell the syrup to local farmers by tanker truck at low margins due to a maintenance issue or other plant imbalances.

Thus, condensed distillers syrup is the least valuable of the three corn ethanol byproducts: DDGS, distillers corn oil, and syrup. Under best circumstances, the syrup's dry weight (approximately 40%) is added to DDGS at market prices or sold at roughly one tenth the price as condensed distillers syrup to farmers for animal feed.

A variety of microbial approaches to valorize bioethanol plant by-products have been proposed, including the production of protein-rich fungal biomass (Bulkan, G., Ferreira, J.A., Rajendran, K. and Taherzadeh, M.J., 2020. Techno-economic analysis of bioethanol plant by-product valorization: exploring market opportunities with protein-rich fungal biomass production. Fermentation, 6(4), p.99.) organic acid production using granular fermentation (Carvajal-Arroyo, J.M., Candry, P., Andersen, S.J., Props, R., Seviour, T., Ganigue, R. and Rabaey, K., 2019. Granular fermentation enables high rate caproic acid production from solid-free thin stillage. Green Chemistry, 21(6), pp. 1330-1339.) and more traditional anaerobic digestion. A key challenge in each of these approaches is the separation of the product chemical from the complex feedstock mixture.

In addition, thin stillage has been used as a feedstock for oleaginous organisms such as the pink yeast Rhodotorula glutinis for the production of biodiesel (See Yen, H.W., Yang, Y.C. and Yu, Y.H., 2012. Using crude glycerol and thin stillage for the production of microbial lipids through the cultivation of Rhodotorula glutinis. Journal of bioscience and bioengineering, 114(4), pp.453-456). Microbial communities have also been used to produce products such as medium chain fatty acids, succinic acid, lactic acid, or caproic acid using thin stillage as a feedstock (See Fortney, N.W., Hanson, N.J., Rosa, P.R., Donohue, T.J. and Noguera, D.R., 2021. Diverse profile of fermentation byproducts from thin stillage. Frontiers in Bioengineering and Biotechnology, 9, p.695306; Carvajal-Arroyo, J.M., Candry, P, Andersen, S.J., Props, R., Seviour, T., Ganigue, R. and Rabaey, K., 2019. Granular fermentation enables high rate caproic acid production from solid-free thin stillage. Green Chemistry, 21(6), pp.1330-1339).

### SUMMARY OF THE INVENTION

Alternatives to petrochemicals produced from bio-derived sources frequently rely on inputs such as oil from soybeans or sugar from corn, beets, or sugar cane, which are also important food crops. Technologies that allow the use of biology to efficiently produce chemicals from non-food carbon sources can be important to building a sustainable chemical supply chain without petroleum.

An approach that interfaces with existing ethanol, distillery, brewery or similar plant infrastructure and continuously converts aqueous waste streams into hydrophobic chemicals in a pristine organic solvent can enable a means for sustainable chemical production with reduced requirements for downstream processing.

The present approach employs biofilm bioreactors (such as, for example, bioreactors described in incorporated US Patent Pub. No. US 2021/0253990 A1) to upscale this renewable carbon source, not currently used for human food consumption and not very valuable otherwise, into valuable organic chemicals such as retinol (Vitamin A) and lubricants. In more detail, such a biofilm bioreactor can use a two-phase method for converting an aqueous feedstock into a hydrophobic product that can be extracted using an immiscible organic solvent, thereby providing a means to isolate the product from aqueous phase contaminants.

The present invention encompasses a method and apparatus for the conversion of thin stillage or other low value streams in corn ethanol plants, distilleries or breweries into hydrophobic chemical products using a biofilm bioreactor system that can be integrated into fermentation plants (corn oil, distilleries, etc.) to ingest fermenter byproduct (e.g., extra-thin stillage, condensed distillers syrup, etc.), and convert these into high value chemicals.

More specifically, described herein, is a bioreactor system that includes one or more biofilm bioreactors that convert an aqueous feedstock into a hydrophobic product that can be extracted into an organic solvent. The bioreactor contains a biofilm made of microorganisms that form a stable biofilm, metabolize the components of the substrate (feedstock), and have a tolerance for organic solvents. Illustrative organisms that can be employed include, for example, Marinobacter species, Pseudomonas species, Chromatiacea spp., and Labrenzia spp, and others.

The biofilm bioreactor comprises a solid phase, support or matrix such as a packed bed, wherein the solid phase includes particles or beads suitable for supporting the biofilm of hydrocarbonoclastic microorganisms. Such a bioreactor has an inlet for the introduction of media, e.g., media containing a carbon source such as thin stillage, which sustains the growth of the organisms of the biofilm. The media is converted into chemical products by biosynthetic pathways contained within the organism.

In some implementations, the invention features a biofilm bioreactor comprising beads for holding (supporting) a biofilm; and oxygen permeable tubing for supplying oxygen content during media recirculation. The design enhances the oxygen supply to the reactor, while reducing, minimizing or eliminating bubble formation.

In some embodiments, a biofilm-forming microbial community is used as the microbial catalyst, where different microbial species can metabolize different components of the thin stillage or fermentation byproduct or catalyze different steps of product formation.

In some embodiments, the organism is tolerant to organic solvents, a property that enables two phase extraction of products from the bioreactor while preserving functional biomass.

Some embodiments of the invention feature a system designed to be integrated in the plant prior to evaporators (used to concentrate thin stillage); the medium employed is compatible with the organism in the bioreactor which then convert that medium into product. A mixing system can be used ahead of the entrance to the bioreactor to introduce crucial components to the medium.

Some embodiments of the invention feature a system designed to use concentrated thin stillage (or condensed distillers solubles (also referred to as “syrup”, abbreviated as “CDS”)) and the reactor is integrated into the plant after one or more evaporation cycles. In this process, a pump can be used to recirculate media through the bioreactor; concentrated thin stillage is incrementally introduced into the bioreactor as carbon is consumed.

Some embodiments of the invention feature a system designed to use distillers corn oil (or a similar stream) as a feedstock, and the reactor is integrated into the plant after the centrifugation step. This process can employ an emulsifier module, e.g., a mixer, at the inlet, to disperse fine corn oil droplets in the media. Prior to product extraction with the organic solvent, the column is flushed with a secondary media stream that does not contain corn oil to minimize cross-contamination of the feedstock extracted product stream. An arrangement in which the system is integrated after the centrifugation step (and before the final drying step) also can employ byproduct left behind after extracting the distillers corn oil.

Also encompassed by the present invention are methods for converting thin stillage, distillers corn oil, or another suitable co-product into hydrophobic chemical products using a platform organism that is hydrocarbonoclastic such as Marinobacter species, or Pseudomonas species, in a biofilm bioreactor. In some embodiments these methods use hexanes, heptanes, dodecane, corn oil, etc. as an extraction solvent. The hydrophobic phase is routed to downstream processing. Any aqueous byproduct generated in the process that can no longer sustain productive biofilm culture is flushed from the system. In some embodiments, this aqueous byproduct is returned to the evaporation stream, while in other embodiments, the aqueous byproduct is evaporated and composted.

In one of its aspects, the invention features a method for preparing a hydrophobic chemical. The method comprises directing a co-product from a biorefinery to a bioreactor system that includes multiple biofilm bioreactors, each biofilm bioreactor containing an organism capable of metabolizing the co-product to produce a hydrophobic chemical; circulating the co-product through at least one of the multiple biofilm bioreactors; and operating the at least one of the biofilm bioreactors in response to assessments from sensors associated with the at least one biofilm bioreactor.

In another aspect, the invention features a bioreactor system comprising: multiple biofilm bioreactors, each biofilm bioreactor containing an organism capable of metabolizing a co-product in a biorefinery (e.g., a corn ethanol plant, a brewery, a distillery) to produce a hydrophobic chemical. Each reactor is configured for circulating the co-product, for introducing at least one additional ingredient and/or for collecting the hydrophobic chemical. Also included in the biofilm bioreactor system are sensors for assessing parameters in each of the biofilm bioreactor and a controller for controlling each of the biofilm bioreactors in response to the assessment of the sensors.

The described system and method can be used for converting the complex, low-value carbon sources that are byproducts of fermentation into useful hydrophobic chemicals that require minimal costly downstream processing. In some implementations, the invention advantageously upscales the lowest-value by-product, thin stillage/syrup, for example, into high-value cosmetic ingredients or lubricants.

Not only can arrangements described herein be integrated into an existing plant (without the need for new construction), but aspects of the invention offer multiple options and flexibility with respect to a retrofit that best meets plant or process goals and/or constraints.

Generally, separating a desired product from an unrefined, complex medium is challenging. By implementing a continuous-flow design such as described herein, a design in which a secondary phase (e.g., a hydrophobic solvent) is used to extract product, feedstock and product can be separated without cross contamination, reducing the burden on downstream processing, often a crucial aspect for a cost-effective production of chemicals and in particular of high-end chemicals.

A design in which a film bioreactor employs oxygen permeable tubing for delivering oxygen-containing gas inside the reactor can reduce, minimize and typically eliminate bubble formation.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram showing an ethanol plant's end-to-end production of their primary target ethanol and by-products animal feed (DDGS), distillers corn oil, and condensed distillers syrup (CDS);

FIG. 2 is a schematic diagram of a biofilm bioreactor in which glass beads coated with biofilm are packed in a column, with the inset showing a representation of a bead coated with a bacterial biofilm;

FIG. 3 is a schematic diagram showing an ethanol plant's end-to-end schematic and the insertion of continuous-flow biofilm bioreactors with hydrocarbonoclastic organisms included after the corn oil extraction and final evaporation step but prior to the final dryer;

FIG. 4 is a schematic diagram showing an ethanol plant's end-to-end schematic and the insertion of continuous-flow biofilm bioreactors with hydrocarbonoclastic organisms included between any step of the multi-evaporator cascade which occurs after the centrifuge separation of thin stillage from whole stillage;

FIG. 5 is a schematic diagram showing an ethanol plant's end-to-end schematic and the insertion of continuous-flow biofilm bioreactors with hydrocarbonoclastic organisms after centrifuge separation of thin stillage from whole stillage, but before the evaporation cascade;

FIG. 6 is a schematic diagram showing a schematic of the biofilm bioreactor system including a bioreactor that recirculates media through the reactor, and includes a valve for the periodic introduction of thin stillage and sensor for monitoring carbon source content;

FIG. 6A is a flow chart for a process conducted in the configuration depicted in FIG. 6;

FIG. 7 is a schematic diagram showing a schematic of a biofilm bioreactor system where a mixer is used to mix salts and minerals with thin stillage and then introduce it into the bioreactor;

FIG. 7A is a flow chart for a process that can be conducted in the configuration depicted in FIG. 7;

FIG. 8 is a schematic of a biofilm bioreactor that has oxygen permeable tubing through the middle of the reactor to increase gas transport while not creating bubbles;

FIG. 9 shows a table of the chemical composition of thin stillage, according to Kim, Y., Mosier, N.S., Hendrickson, R., Ezeji, T., Blaschek, H., Dien, B., Cotta, M., Dale, B. and Ladisch, M.R., 2008, Composition of corn dry-grind ethanol by-products: DDGS, wet cake, and thin stillage. Bioresource technology, 99(12), pp.5165-5176.);

FIG. 10 is a plot of relative fluorescence units (RFU) as a function of time in hours; and

FIG. 11 is the UV-vis absorbance spectra of solvent overlays collected from retinoid-producing M. atlanticus cultures grown on corn mash at different concentrations with and without glycerol

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an,” and “the” do not preclude plural referents, unless the content clearly dictates otherwise.

As used herein, the term “about” when used in conjunction with a stated numerical value or range denotes somewhat more or somewhat less than the stated value or range, to within a range of ±10% of that stated.

In some of its aspects, the invention features a bioreactor system that includes at least one biofilm bioreactor containing an organism that can metabolize one or more components of a co-product stream. The bioreactor system can further include connections (valves, conduits, etc.) to introduce a co-product stream (or other ingredients) into the bioreactor(s) and/or connection (valves, conduits, etc.) for withdrawing contents (e.g., product, waste). Pumps, reservoirs, mixing devices, lines for supplying air or other ingredients, or other devices also can be included.

In some implementations, the co-product stream is a stream generated in a corn ethanol plant. Embodiments of the invention apply, however, not only to corn ethanol plants but also to distilleries, breweries or other types of biorefineries. Thus, the invention can be practiced with processes and/or plants using fermentation feedstocks such as: Corn (maize), Sorghum (milo), Barley, Rye, Oats, Wheat, Soybeans, Rice, Millet, Sugarcane, Sugar Beets, Grapes, Agave, Apples, Apricots, Potatoes, Beets, Honey, Milk, Walnuts, Cashews, Peanuts, Pecans, Buckwheat, Sap of Palm, Sweet Potato, Ginger, Triticale, Cassava, Guarana, Coconut, Cherries, Blueberries, Raspberries, Pomegranate, Pineapples, Pears, Plums, Bananas, Plantains, Juniper Berries, Sunflowers, Rapeseed (and other oilseeds). These and other similar feedstocks can be processed at biorefineries which include, but are not limited to, those producing Beer, Wine, Cider, Mead, Sake, Kefir, Kombucha or similar beverages, in addition to biorefineries known as distilleries producing distilled spirits such as, for example, Brandy, Gin, Rum, Vodka, Tequila (or Mezcal), and all types of Whiskeys to include Scotch, Rye, Bourbon, Irish, Canadian, and Japanese Whiskeys and the like.

In one example, the bioreactor system is used in the conversion of stillage and in particular thin stillage, to a hydrophobic product. In other examples, the bioreactor system is used in the conversion of distillers com oil (or distillers condensed solubles) into a hydrophobic product.

In general, “stillage” refers to the remaining mash, or mixture of liquid with fermentation byproducts and unfermented suspended solids resulting from the biorefineries. While the trade name for this mix of liquid with unfermented solids changes among various industries, countries, and even regions, embodiments described herein apply to all stillage byproducts from biorefineries and feedstocks listed above as well as other (similar) biorefineries. The composition of an illustrative thin stillage co-product is presented in FIG. 9. Distillers corn oil (DCO), also referred to herein as “corn oil” is a co-product of corn ethanol production, generated (e.g., by centrifugation) from CDS. Typically, it contains higher amounts of fatty acids relative to many other vegetable oils and can be used as biodiesel feedstock or poultry feed ingredient. “Condensed (concentrated) distillers solubles” or “distillers condensed (concentrated) solubles” (syrup) or CDS is a low fiber, high protein product that is also rich in organic acids derived from the ethanol production process.

Aspects of the invention are specifically designed for the scalable production of hydrophobic chemicals Separating cell biomass from the fermentation broth, extracting product from the cells, and then separating the product from the unwanted cellular debris makes hydrophobic chemicals particularly hard to synthesize via large-scale fermentation. Using a microorganism having a tolerance to hydrophobic organic solvents makes possible an in situ, continuous-flow product extraction. In this method, an immiscible organic solvent is pulsed through the reactor to extract the hydrophobic product, while not harming the biofilm.

Other properties may play an important role. Accordingly, the organism employed in practicing aspects of the invention provides one and typically more than one desirable features and/or functions. It can form biofilms, can metabolize the primary carbon components of thin stillage, distillers oil, etc., has a tolerance to organic solvents, and, in some cases, can be engineered to for desirable conversion pathways.

Illustrative organisms that can be used in the biofilm bioreactor include, for example, Marinobacter species, Pseudomonas species, Chromatiacea spp., and Labrenzia spp. Both Marinobacter spp. and Pseudomonas spp. are generally genetically tractable (See Bird, L.J., Wang, Z., Malanoski, A.P., Onderko, E.L., Johnson, B.J., Moore, M.H., Phillips, D.A., Chu, B.J., Doyle, J.F., Eddie, B.J. and Glaven, S.M., 2018, Development of a genetic system for Marinobacter atlanticus CP1 (sp. nov.), a wax ester producing strain isolated from an autotrophic biocathode, Frontiers in microbiology, 9, p.3176.) and are well known for their opportunitroph behavior, characterized by the ability to utilize a wide variety of feedstocks (carbon sources), and tolerate a variety of stressors, including solvent exposure (See Ramos-González, M.I., Ben-Bassat, A., Campos, M.J. and Ramos, J.L., 2003, Genetic engineering of a highly solvent-tolerant Pseudomonas putida strain for biotransformation of toluene to p-hydroxybenzoate, Applied and environmental microbiology, 69(9), pp.5120-5127; Klein, B., Bouriat, P., Goulas, P. and Grimaud, R., 2010, Behavior of Marinobacter hydrocarbonoclasticus SP17 cells during initiation of biofilm formation at the alkane-water interface, Biotechnology and bioengineering, 105(3), pp.461-468).

Some embodiments employ organisms such as, for instance Marinobacter species and Pseudomonas species as biofilm-forming hydrocarbonoclastic microorganisms, sometimes referred to as “hydrocarbon degrading” microorganisms (e.g., bacteria) or “oil degrading” microorganisms (e.g., bacteria). Specific examples include: Marinobacter spp., and in particular Marinobacter atlanticus; a Marinobacter spp. such as M. psychrophilus (See Zhang, D.C., Li, H.R., Xin, Y.H., Chi, Z.M., Zhou, P.J. and Yu, Y., 2008, Marinobacter psychrophilus sp. nov., a psychrophilic bacterium isolated from the Arctic, International Journal of Systematic and Evolutionary Microbiology, 58(6), pp.1463-1466); Marinobacter sp. LV10R520-4, Marinobacter sp. LV10MA510-1, Marinobacter sp. ELB17, and other similar Marinobacter spp. (See Cooper, Z.S., Rapp, J.Z., Shoemaker, A., Anderson, R.E., Zhong, Z.P. and Deming, J.W., 2022, Evolutionary divergence of Marinobacter strains in Cryopeg brines as revealed by pangenomics, Frontiers in Microbiology, p. 1883).

In some cases, the organisms natively possess all or some of the genes required to produce a desired product, retinal or retinol for example In others, they can be engineered with a pathway to convert carbon building blocks such as acetyl-CoA into more complex chemical products, such as, for instance, isoprenoids and retinol. Genetically-engineered organisms comprising synthetic operons for the production of isoprenoids, carotenoids, and retinoids, optimized for use in a hydrocarbonoclastic organism, and methods for the synthesis and extraction of isoprenoids in a biofilm bioreactor comprising the genetically-engineered organisms are described by Magyar et al. in U.S. Patent Application Publication No. US 2022/0340949 A1, published on Oct. 27, 2022 and incorporated herein in its entirety by this reference. According to some aspects of the present invention, principles described in the US 2022/0340949 A1 are adapted for the conversion of a low-value co-product from a corn ethanol process or from distillery/brewery waste to useful chemicals

Some implementations employ a biofilm-forming microbial community, used as the microbial catalyst, where different microorganism species can metabolize different components of the thin stillage or fermentation byproduct or catalyze different steps of product formation.

While the alcohol (ethanol, for example) is the product of corn ethanol plants or other biorefineries, operations associated with the alcohol production generate various co-product streams. Embodiments of the invention relate to the use of such a co-product stream to prepare chemicals.

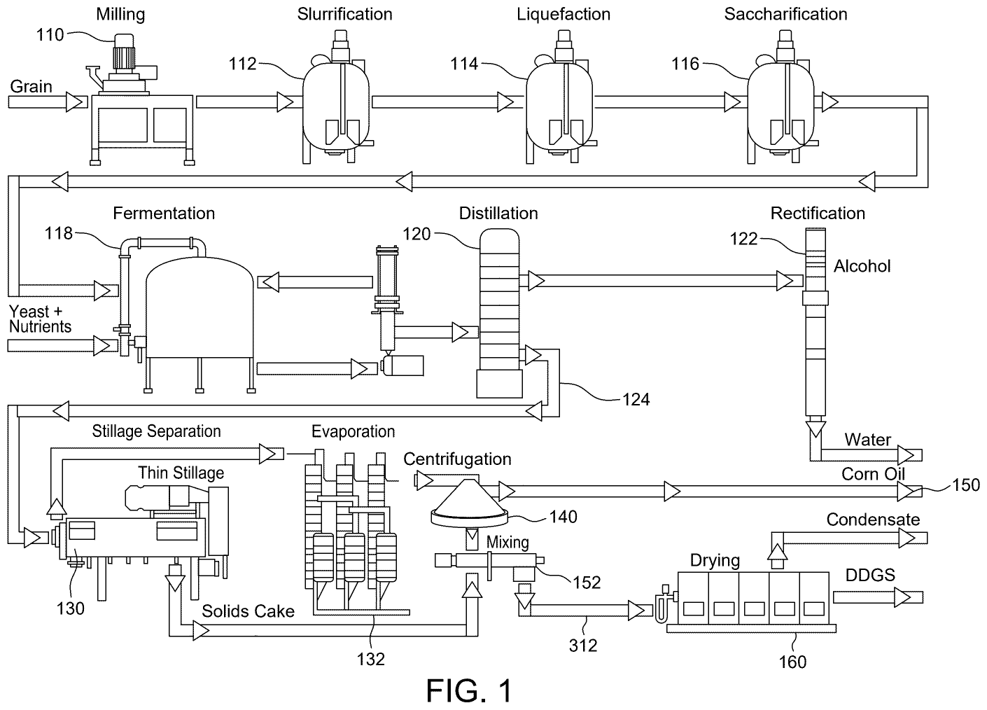

As known in the art, corn ethanol plants can employ any number of process schemes. FIG. 1 presents an illustrative overview of a dry-mill corn ethanol plant for ingesting corn from American farmers by mixing with yeast, water, and enzymes to convert the available corn starch into sugar. Subsequent steps yield ethanol and other byproducts.

The dry-mill plant in FIG. 1 includes milling stage 110 to dry-mill the corn, using, for example, a hammermill. A slurrification stage 112 creates a slurry. A liquefaction stage 114 mixes corn with water and enzymes (e.g., amylolytic enzymes for yeast). A saccharification stage 116 converts, by the enzymes, starches into sugars and dextrins. A fermentation stage 118 ferments the product from the saccharification stage using added yeast and nutrients. A distillation stage 120 distills the alcohol in a series of distillation columns, the remaining byproduct called “whole stillage” (124), typically comprises water, fiber, protein and oil.

From the distillation columns, the alcohol (e.g., ethanol) is directed to a rectification stage 122 to produce concentrated alcohol. The whole stillage 124, containing both liquid and suspended solids left-over from fermentation, enters a stillage separation stage 130 that can employ centrifugation to separate thin stillage from solids. From the separation stage 130, the extracted wet cake is sent to mixer 152 (or collected) as DDG. Thin stillage from separator stage 130 is directed to an evaporator stage dryer (e.g., dryer cascade 132) to form condensed distillers solubles (or syrup), CDS, which passes to centrifuge 140 to extract distillers corn oil (line 150). From centrifuge 140, the remaining thin stillage (depleted in oil) is combined with wet cake in mixer 152 and the resulting mixture is passed to a final drying stage 160 for producing condensate and Dried Distillers Grains with Solubles (DDGS). In a different process scheme, liquid left over after the extraction of the distillers corn oil in centrifuge 140 is dried (e.g., by evaporation), to generate distillers solubles that are then sent to the drier to produce DDGS (with reduced fat content). Other plant/process arrangements can be employed, depending on desired byproducts or other considerations.

According to some aspects of the invention, one or more biofilm bioreactor(s) can be integrated in a corn ethanol plant (such as that shown in FIG. 1), in a distillery, a brewery or in another biorefinery. More specifically, the one or more biofilm bioreactors can be inserted on a co-product (also referred herein as “by-product”) line (the “product” of a biorefinery such as a corn ethanol plant, brewery or distillery being the alcohol).

In one example, the co-product is thin stillage, a complex mixture of components ˜30% of which is glycerol and lactic acid (Kim, Y., Mosier, N.S., Hendrickson, R., Ezeji, T., Blaschek, H., Dien, B., Cotta, M., Dale, B. and Ladisch, M.R., 2008, Composition of corn dry-grind ethanol by-products: DDGS, wet cake, and thin stillage, Bioresource technology, 99(12), pp.5165-5176.). The thin stillage mixture can be metabolized by platform organisms such as described above in the biofilm bioreactor, the biofilm state providing the microorganisms the resilience to withstand components of thin stillage (acetate and glycerol, for instance) that can inhibit non-biofilm forming organisms, such as E. coli (Pinhal, S., Ropers, D., Geiselmann, J. and de Jong, H., 2019, Acetate metabolism and the inhibition of bacterial growth by acetate, Journal of bacteriology, 201(13), pp. e00147-19.)

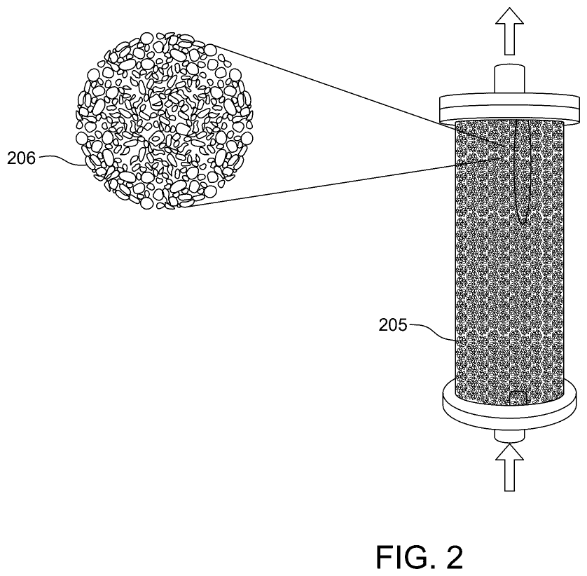

FIG. 2 shows a biofilm bioreactor 205 as described in Glaven. It provides a robust platform for making chemicals that have traditionally been petrochemicals. Biofilms are naturally-forming communities of microorganisms that adhere to each other and to surfaces of beads 206 in the reactor 205, providing protection from the environment around them. The flexible metabolism and robustness of biofilm-forming organisms such as M. atlanticus (or others, described above) allows access to a wide range of feedstocks inaccessible to traditional yeast or E. coli fermentation, including ethanol plant co-products such as thin stillage.

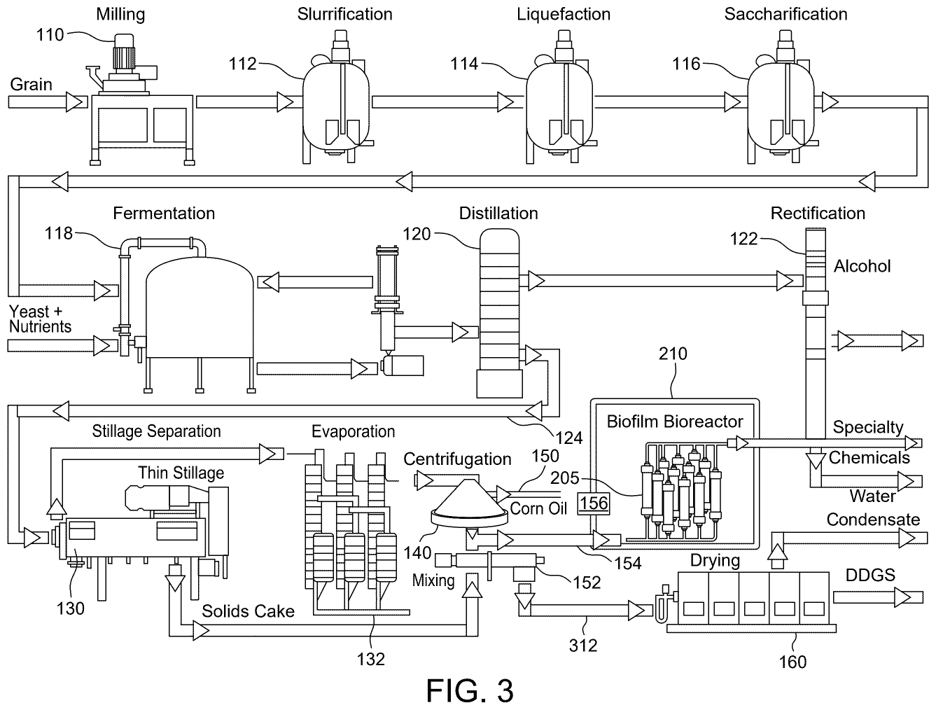

Many embodiments utilize multiple (at least two) continuous-flow biofilm bioreactors 205 connected in parallel to reduce the threat of a single reactor contaminating the entire series. A parallel arrangement also ensures a more equal distribution of nutrients. In general, the effluent or waste stream from the biofilm bioreactors can be: 1) returned to the ethanol plant pipeline at the point of its extraction (from the co-product line 154 (see FIG. 3, further described below); 2) returned to the final step of thin stillage/syrup processing, the final gas dryer (drying stage 160) leading to the DDGS product line; or 3) landfilled as waste and not returned to the ethanol plant. There are benefits and disadvantages for each effluent management step, as further discussed below.

In many implementations, a biofilm bioreactor assembly or array (containing one and typically more than one bioreactors, e.g., in a parallel configuration) are inserted downstream of the line extracting the product, e.g., downstream of the distillation/rectification stage 120 of the ethanol plant illustrated in FIG. 1. For instance, the insertion can be made downstream of the stillage separation stage 130.

The co-product, thin stillage, condensed distillers syrup, or distillers corn oil, for instance, is fed into the bioreactor from the bottom and flows up against gravity. It is passed through a bioreactor multiple times by a recycling system that can be adjacent to each modular bioreactor. Culture media, nutrients, salts and/or other ingredients are added to sustain the biofilm microrganisms. The depleted thin stillage or syrup is eventually released back to the ethanol pipeline or sent to landfill waste. During or following this step, the hydrophobic target molecule is “harvested” from the continuous flow biofilm bioreactors by circulating an organic solvent through the reactors. The target molecule containing solvent is then passed through a separation process such as column chromatography or nanofiltration to purify the target molecule and regenerate the organic solvent (which can be returned to the reactor or discarded). Specific embodiments employ the organic solvent hexane or distillers corn oil, already purified and stored at the ethanol plants. With the organic solvent hexane used in small scale systems, evaporative separation of the target molecule from hexane is also possible. Heptanes, dodecane, other hydrocarbons, vegetable (e.g., corn) oils, other hydrophobic organic solvents, mixtures of solvents, etc. also can be used.

Typically, the continuous-flow biofilm bioreactor assembly taps into the higher pressure co-product line, e.g., thin stillage or syrup line, to draw an initial flow of nutrient rich liquid into the recirculating media with buffered pH, salt, and micronutrient content for the hydrocarbonoclastic organism. This flow can either be assisted with pumps or solely rely on the high pressure from the co-product line to push both the initial and recirculating nutrient flow, by leveraging well-positioned and well-timed one-way valves. The recirculating system slowly “bleeds” or “drips” off this tapped ethanol plant co-product, e.g., thin stillage or syrup, line every “cycle” of the fluid to replenish consumed nutrients. This is an important advantage of incorporating a continuous flow bioreactor inside an ethanol plant.

FIG. 3 shows an embodiment in which parallel-connected array 210 of continuous-flow biofilm bioreactors 205, containing a hydrocarbonoclastic organism (such as described above, for instance) is inserted at line 154 after both the corn oil extraction and final evaporation step 132 but prior to the final dryer (drying stage 160). This arrangement has the advantage of accessing the most condensed liquid nutrient stream for the “bleed” or “drip” process described above, in addition to reducing the potential for ethanol plant equipment damage in the case of reinsertion of spent media.

In the approach shown in FIG. 3, the only equipment that will process the added salts and other micronutrients of the hydrocarbonoclastic organism's growth media is the gas dryer at drying stage 160. Furthermore, a placement after the oil extraction process does not impact that valuable co-product in any way, either by reducing its quantity (which can happen if the bioreactors are inserted upstream and there is no reinjection of bioreactor waste), or the quality (if there is no re-injection of waste and the added salts or nutrients reduce the oil extraction efficiency or equipment “up-time” of the extraction centrifuge). Hydrocarbonoclastic organisms may also metabolize the corn oil directly thus partially consuming a valuable co-product.

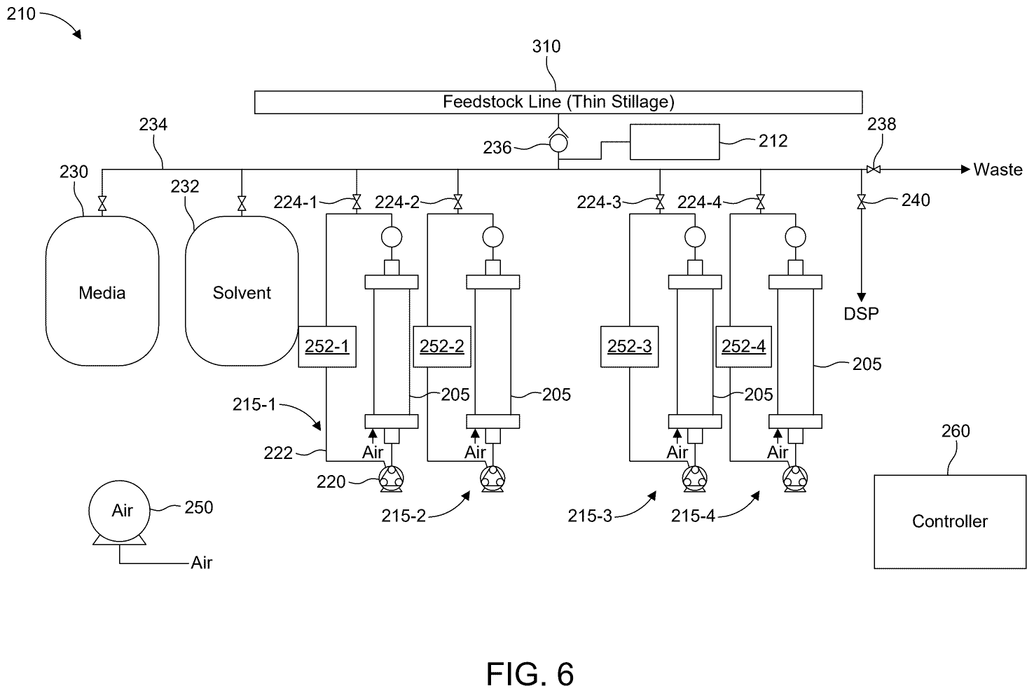

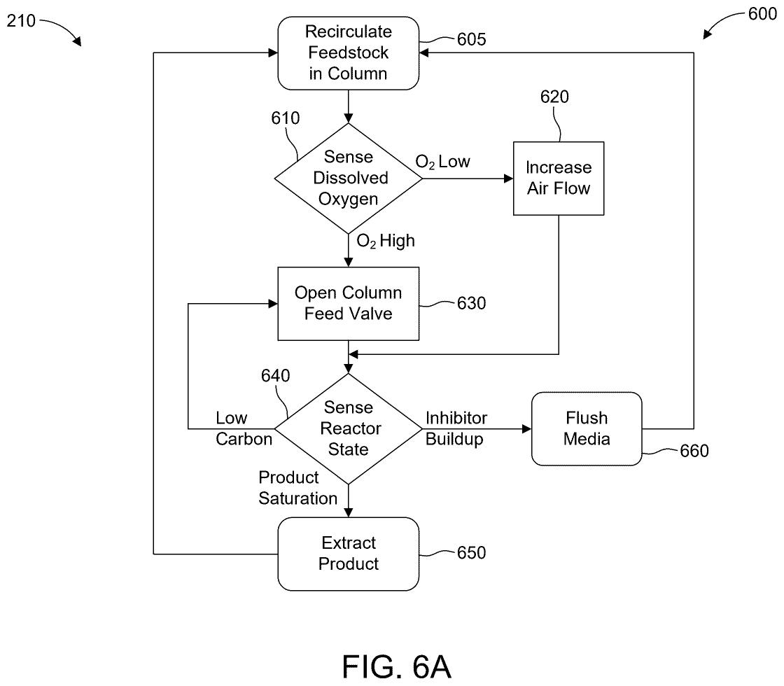

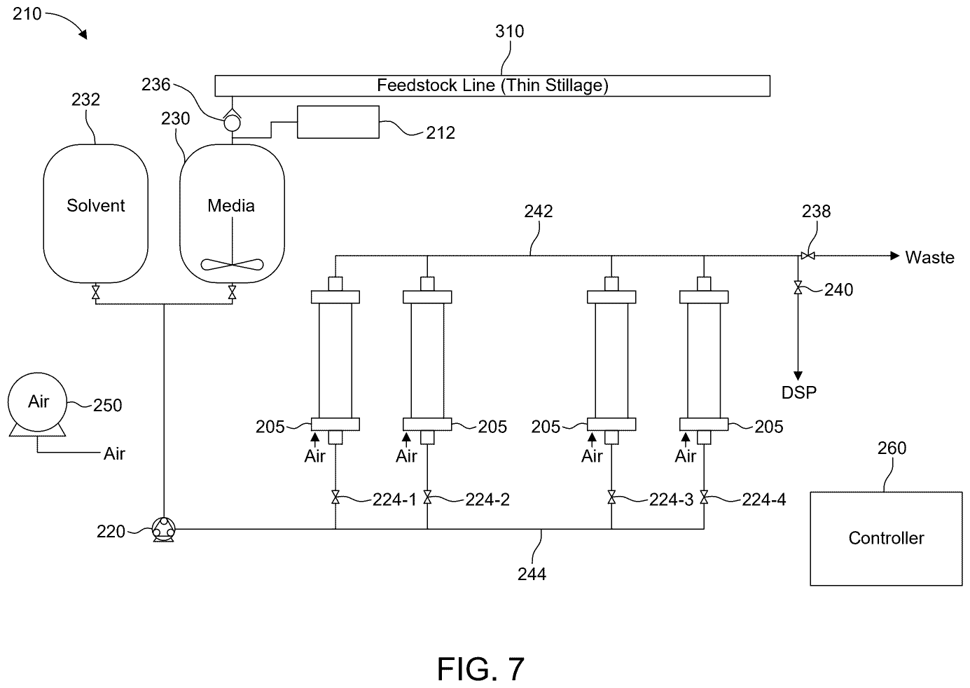

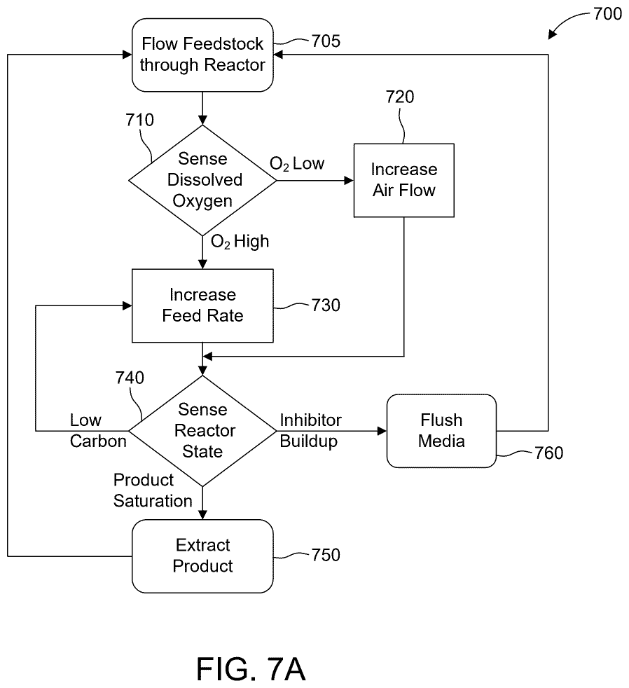

In the embodiment of FIG. 3, a sampling (feed) valve (see, e.g., valve 236 in FIGS. 6 and 7, further described below) at line 154 is used to introduce thin stillage feedstock from centrifuge 140 into a pre-prepared medium (see, e.g., FIG. 7, further described below) or to a film bioreactor, after mixing with salts, etc. (see, e.g., FIG. 6, further described below). In some embodiments, this sampling valve is controlled by a sensor (disposed, for instance, inside the bioreactor or in-line with the bioreactor) that monitors the level of lactate, glycerol, or another metabolizable component of the medium. The valve can be programmed to introduce the condensed nutrient stream into the recirculating media when the sensor detects that the carbon-source level has dropped below a critical threshold. In some embodiments, the feedstock to array 210 is distillers corn oil (from line 150). An emulsifier module 156 can be used to introduce corn oil droplets into the reactor as the carbon source.

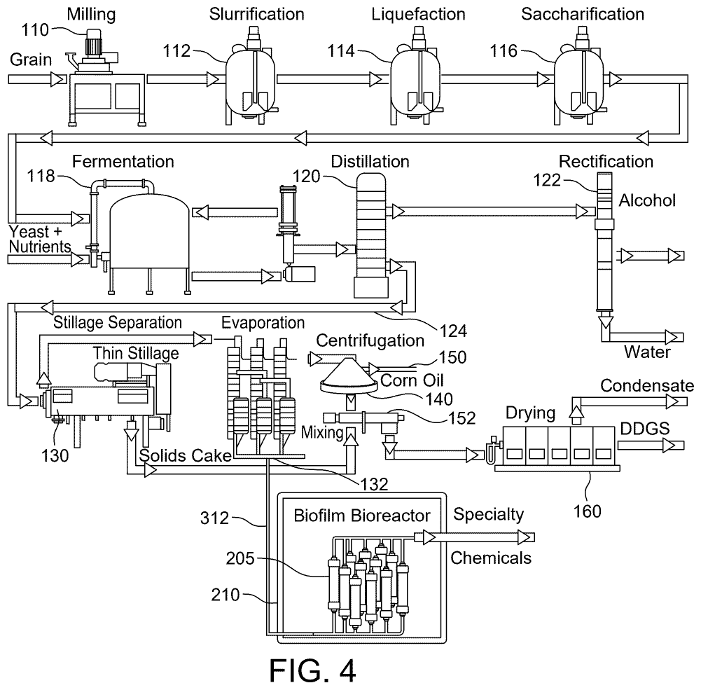

FIG. 4 shows another embodiment in which the continuous-flow biofilm bioreactor array 210 containing a hydrocarbonoclastic organism in the bioreactors 205 are inserted between any step of the multi-evaporator cascade 132 which occurs after the (centrifuge) separation of thin stillage from whole stillage (stage 130), but before the corn oil centrifuge extraction (stage 140). This allows for fine tuning the thin stillage concentration best suited for the recirculating nutrient mix. Most dry mill ethanol plants have at least a half-dozen or more continuous-flow evaporators which transition the raw thin stillage from roughly 90% water down to about 60% water. Being able to insert array 210 at a selected location in the cascade series allows access to a finely-tuned thin stillage concentration. In some implementations, the co-product used in array 210 is distillers corn syrup (CDS). As noted above, however, this can negatively impact the oil extraction step; however, the oil molecules present before the corn oil extraction can also serve as a carbon source for the organism.

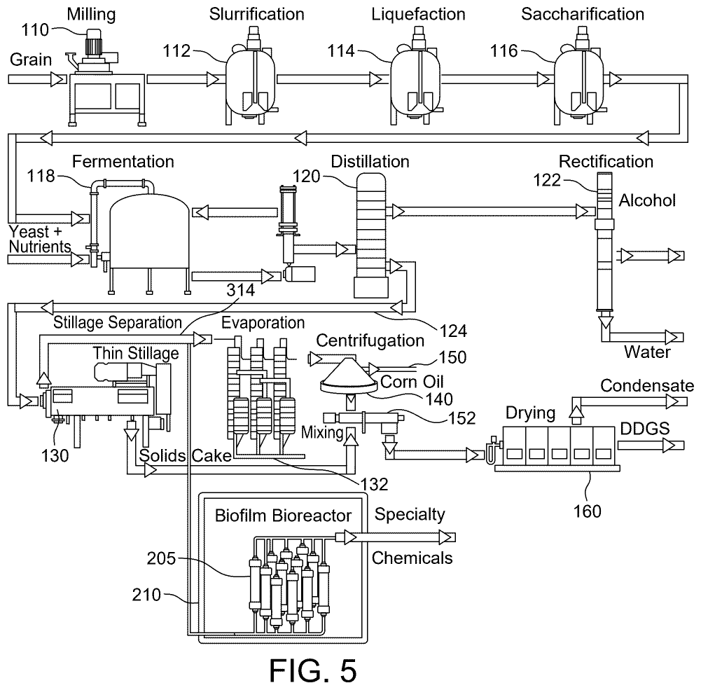

FIG. 5 shows a further embodiment in which the continuous-flow biofilm bioreactor array 210, containing a hydrocarbonoclastic organism, is inserted after centrifuge separation of thin stillage from whole stillage, but before the evaporation cascade. This reduces the amount of energy consumed by the ethanol plant to evaporate the thin stillage but can negatively impact oil extraction. It also yields the most dilute nutrient source thus complicating the “bleed” or “tap” access schemes described herein in addition to a likely increase in down-time for cleaning all downstream equipment if effluent waste is re-injected. In some versions of this embodiment, a mixer (further described below) is used to add required salts, minerals, and other media components to the diluted thin stillage prior to the medium entering the bioreactor.

The biofilm bioreactor array and its operation are further described below.

FIG. 6 shows one implementation of the continuous-flow biofilm bioreactor array 210. As presented in FIG. 6, each of the bioreactors 205 is isolated in a separate loop 215-1 to 215-4. Specifically, each loop 215-1 to 215-4 comprises a circulation pump 220 controlled by controller 260 along with a return pipe 222. A loop valve 224 controlled by controller 260 enables introduction of new material into each loop and the harvesting of material from that loop. Arrays can contain one or more loops, e.g., within a range of from about 1 to about 100, such as 1 through: 5, 10, 20, 30, 40, 50, 60, 70, 80 or 90. One example employs 10 loops.

A growth media reservoir 230 and a solvent reservoir 232 provide growth media and solvent to the loops 215-1 to 215-4 via a manifold line 234 via respective valves controlled by controller 260. A feed valve 236 controlled by controller 260 supplies feedstock to manifold line 234 for the periodic introduction of co-product, e.g., thin stillage. A mixer 212 controlled by controller 260 can be used to supply salts, minerals, and other media components to the co-product, e.g., diluted thin stillage, prior to the medium entering the bioreactor. Sensors 252-1 to 252-4 for enabling the controller 260 to monitor carbon source content. The controller uses the sensors to determine when to open the valve 224-1 to 224-4 for the injection of the thin stillage can be located inside each bioreactor or in each of the recirculation lines, namely loops 215-1 through 215-4.

Air can be supplied from a suitable source. In one implementation, it is introduced via a line to the bottom of each bioreactor 205 and is generated by a compressor 250.

In this implementation the state of the bioreactor is continuously monitored by the controller via sensors 252-1 to 252-4 (either within the bioreactor columns or in line with each column). These sensors are typically electrochemical and may measure pH, dissolved oxygen, dissolved CO₂, specific carbon sources such as glycerol or fatty acids, or the concentration of the product, Vitamin A, for example. In some cases, the sensors are optical, measuring cell density through light scattering, total product by UV-Vis absorbance, or molecular profiling with Raman or IR spectroscopy. In some instances, the sensor will measure electrical impedance where the electrical impedance spectrum can provide a fingerprint to assess the state of the bioreactor, providing information about cell health, feedstock availability, and product accumulation.

Waste is output from the manifold line via control of a waste valve 238. Product is withdrawn (collected) via control of a product valve 240. In one example, the product is retinol, retinaldehyde, and/or retinoic acid.

In specific implementations, the bioreactor system is controlled by the controller 260 e.g., a microcontroller, computer system, microprocessor, etc., that will monitor the bioreactor state and specifically the described sensors and open and close individual valves to maintain productivity in the reactor. The controller will implement an algorithm where feedstock is continuously recirculated in each column. Dissolved oxygen is detected (by sensors) at the outlet of each column and air flow (supplied from a suitable source) can be adjusted (under instructions from the controller) to compensate. If dissolved oxygen is too high, it is indicative of decreased cellular metabolism. Feed valve 236 is opened as the controller responds to this assessment.

The reactor state can be monitored either through characterizing the electrical impedance inside the bioreactor or through an assessment of all the attached sensors. If the state assessment indicates low carbon, the feed valve is opened by the controller. If the assessment by the controller is that inhibitory byproducts have accumulated, the media is flushed to waste, and fresh media is introduced into the reactor. If the assessment is that sufficient product has accumulated for extraction, the biomass is exposed to the organic solvent. Upon the completion of these actions, the controller returns the reactor to closed loop recirculation within the column.

FIG. 6A is an illustrative flow chart describing the operation, monitoring and control of a process conducted in the arrangement of FIG. 6 by the controller 260. The procedure 600 includes recirculating feedstock (step 605). Parameters of this operation are assessed (using one or more suitable sensors disposed inside the bioreactors 205 or in one of the loops 215-1 through 215-4) and the information is sent to controller 260 which can maintain the status quo or instruct the activation of valves, pumps, change in flow rates or the performance of other actions. For example, the status of dissolved oxygen is evaluated at step 610. If the determined oxygen level is low, the controller instructs an increase in air flow (step 620). In response to a high oxygen level, column feed valve 236 can be opened (step 630), typically under instructions from the controller, to add feedstock (e.g., thin stillage). The reactor state can be evaluated by a suitable sensor (step 640). A determination of low carbon levels can trigger the controller to instruct the opening of the column feed vale 236 (step 630). Response to product saturation by the controller will typically involve product collection or extraction (step 650). The remaining fraction is returned to the recirculating feedstock in the column (any of loops 215-1 through 215-4). Inhibitory byproduct buildup will trigger step 660 in which the controller instructs the flushing of existing media and the addition of fresh media to the recirculating feedstock in the column (one of the loops 215-1 through 215-4).

FIG. 7 shows another mode of arranging and operating the continuous-flow biofilm bioreactor array 210.

Here, the co-product, e.g., thin stillage, is introduced (see valve 236) into media reservoir 230 that is provided with mixer 212 for mixing salts, minerals, etc., with thin stillage, for introduction into the bioreactor array 210 under the control of controller

A single pump 220 feeds the solvent (from reservoir 232) and media (from reservoir 230) to bioreactors 205, connected in parallel. Separate input valves 224-1 to 224-4 control the flow into the bioreactors 205 from an input manifold 244. An output manifold 242 connects the bioreactors to the waste and product outputs (through valves 238 and 240, respectively).

Air can be supplied from a suitable source. In one implementation, it is introduced via lines to the bottom of each bioreactor 205, which lines are fed by compressor 250.

In this implementation the state of the bioreactor is also continuously monitored by sensors (as described with reference to FIG. 6) either within the bioreactor columns or in line with the reactor outlet enabling feedback control by the controller.

As with the embodiment of FIG. 6, the bioreactor system will be controlled by the controller 260 that will monitor the bioreactor state and open and close individual valves to maintain productivity in the reactor. The controller will implement an algorithm where feedstock is continuously fed through the reactor system. It will respond to the assessment of various parameters essentially as described above. Dissolved oxygen is detected at the outlet of each column and air flow is adjusted (under the control of controller 260 to compensate. For instance, if dissolved oxygen is too high, this determination is indicative of decreased cellular metabolism, and prompts the controller to take steps towards a feed rate increase. The reactor state is detected either through characterizing the electrical impedance inside the bioreactor or through an evaluation of all the attached sensors. If the reactor state assessment indicates low carbon, the assessment triggers controller 260 to control opening of feed valve 236. An assessment that inhibitory byproducts have accumulated will result in the media being flushed to waste, and fresh media being introduced into the reactor, both operations being under the control of controller 260. If the assessment is that sufficient product has accumulated for extraction, the controller triggers exposing the biomass to the organic solvent. Upon the completion of these actions, the controller returns the reactor to continuous feedstock flow through the reactor.

A flow chart for the operation described with reference to FIG. 7 is presented in FIG. 7A. In procedure 700, feedstock, passing through any of bioreactors 205 (step 705) is assessed (using a suitable sensor disposed inside one of the bioreactors 205 or in-line to the reactor for dissolved oxygen (step 710). If the controller determines that the oxygen level is low, air flow is increased (step 720). In response to a high oxygen level determination, the media feed from reservoir 230 is increased (step 730). The reactor state is evaluated at step 740. A determination of low carbon levels can trigger an increase in feed rate (step 730). Response to product saturation will typically involve product extraction (step 750). The remaining fraction is returned to the recirculating feedstock. A buildup in inhibitory byproducts will trigger step 760 in which existing media is flushed and fresh media is added to the flowing feedstock. At least some and typically all these determinations and/or subsequent actions are controlled by controller 260.

When integrated in a corn ethanol plant, brewery, distillery, etc., a biofilm bioreactor array arrangement such as that of FIG. 6 or 7 can be connected to a co-product conduit (generally labeled 310 in FIGS. 6 and 7) using techniques and equipment (piping, valves, flowmeters, sensors, etc.) as known in the art.

In the embodiment described with reference to FIG. 3, for example, line 310 receives feedstock from a thin stillage conduit exiting centrifuge 140, at a point upstream of mixing stage 152, for example. Also possible is a connection to distillers corn oil line 150. In another integration arrangement (see, e.g., the embodiment described with reference to FIG. 4), line 310 corresponds to or connects to CDS line 312 exiting the evaporator cascade 132. Further embodiments allow configurations in which line 310 is located at the exit of a specific evaporator selected from the evaporator cascade 132, to better control the composition of the co-product fed to line 310. For the integration approach described with reference to FIG. 5, line 310 corresponds to or connects to conduit 314 which supports the flow stream exiting stillage separation stage 130.

As already noted (with reference to FIGS. 6A or 7A, for instance) supplemental oxygen can be added to the recirculating fluid to increase the hydrocarbonoclastic organism's efficiency to produce the target molecule. In one embodiment, continuous flow oxygenation to the biofilm bioreactor includes the use of oxygen permeable tubing with ambient or compressed air to passively influx oxygen through the tubing wall. In some cases, this can be in addition to the use of active oxygen bubblers within the recirculating lines. It is noted that any active solutions involving in-line oxygen bubblers will typically require bubble traps prior to entry into the bottom of the biofilm bioreactor.

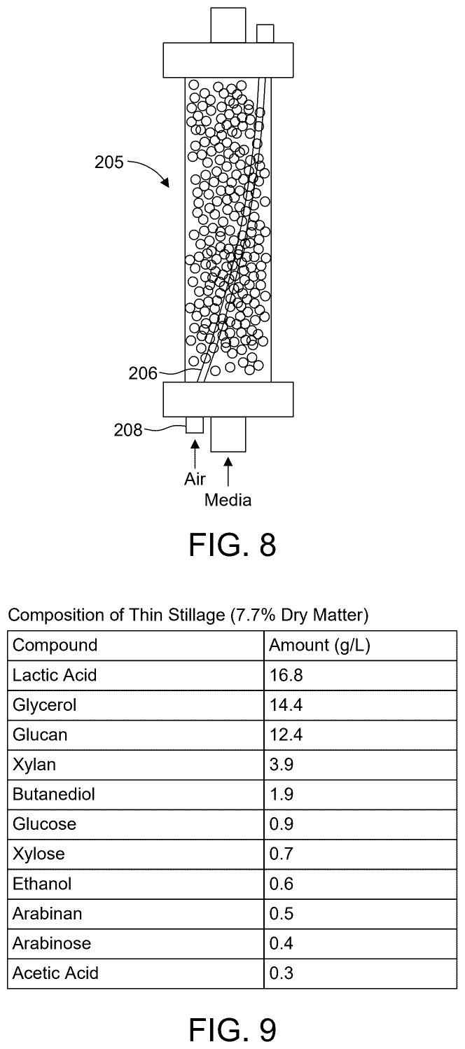

Shown in FIG. 8 is an embodiment in which biofilm bioreactor 205 includes oxygen permeable tubing 206, a fluoropolymer tubing, for example. Other oxygen permeable tubing or tubing materials that can be employed include but are not limited to fluoroethylenepropylene, low density polyethylene, silicone.

Tubing 206 receives air from an air inlet port 208 and extends through the interior of reactor 205, to an air outlet port 214. Air inlet port 208 and air outlet port 214 are constructed, respectively, in bioreactor flanges 216 and 218. The flanges can also include media inlet 252 and media outlet 254. In one implementation, the inlet and outlet ports are arranged in a staggered configuration relative to one another. As a result, tubing 206 is not parallel to the vertical axis or the bioreactor. Other arrangements, e.g., spiral tubing, coiled tubing, multiple tubes connecting pairs of inlet and outlet ports, etc. also can be employed. Oxygen gas or oxygen-enriched air can be used in addition or as an alternative to air In an embodiment such as that of FIG. 8, the gas tubing can increase gas (e.g., air) transport in the bioreactor without generating gas bubbles.

### EXAMPLES

### Example 1: Metabolism of Glycerol and Thin Stillage by M. atlanticus

The two predominant components of thin stillage are lactic acid and glycerol (FIG. 9). Marinobacter sp. are known to use a wide range of organic acids including lactic acid as a carbon source. To evaluate the ability of M. atlanticus to use glycerol as a carbon source, an artificial seawater medium was prepared with glycerol as the sole carbon source at concentrations of 0.8%. 4%, and 8% Each of these samples exhibited significant M. atlanticus growth, indicating its utilization as a carbon source.

To further examine the growth of M. atlanticus on thin stillage as a carbon source, a metabolic assay was carried out on thin stillage samples. Samples of 1 wt % thin stillage from both before and after different evaporator stages were prepared in artificial sea water media optimized for biofilm growth. A 24 well plate was prepared containing silicon dioxide beads that would serve as the solid support. 1 mL of ASW medium containing 1% thin stillage, Evap, or Corn Oil were added per well. Each well was inoculated with 1:100 WT Marinobacter (10 uL) and sealed. The plate was incubated at 30 C for about 7.5 hrs of growth to seed the beads with biofilm. The media was removed and rinsed with ASW containing no carbon.

To evaluate the amount of biofilm that had formed on the beads a metabolic assay was then carried out. ASW containing succinate as a carbon source was added along with resazurin. As a result of metabolic activity, resazurin becomes reduced yielding a fluorescent molecule. The metabolic activity correlates with the total biomass, providing evidence of the relative initial biomass that was on the silicon dioxide beads.

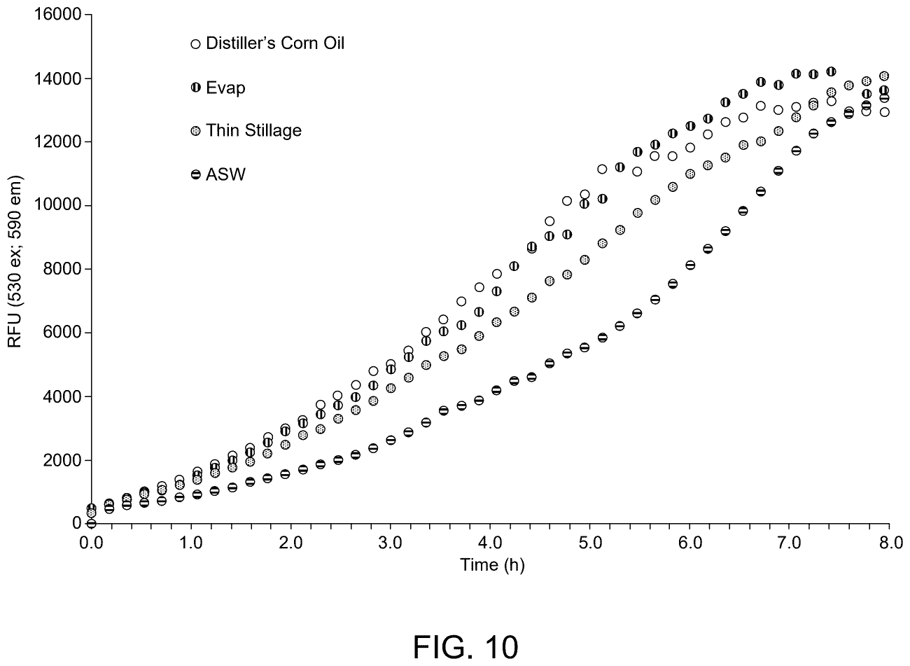

The fluorescence signal (excitation wavelength 530 nanometer (nm); emission wavelength 590 nm) was monitored in the plate reader for more than 10 hours. A more rapid increase in fluorescence signal is indicative of more initial biomass. As indicated in the data shown in FIG. 10, each of the byproducts of ethanol fermentation yielded more biomass than the organic acid (succinate) containing ASW control media. These data indicate that not only is M. atlanticus able to metabolize the components of thin stillage and distiller's corn oil and also from distillery/brewery waste, but that these carbon sources have the potential yield biofilms having more biomass than a minimal media with a short chain organic acid.

Example 2 Production of Retinoids with M. atlanticus Grown on Distillery Corn Mash Waste

To demonstrate the ability to produce retinoids from thin stillage, a strain of M. atlanticus engineered to produce retinal by knocking out the wax ester carbon storage pathway, ΔΔ M. atlanticus (Bird, et al 2018). Subsequently two plasmids were engineered into M. atlanticus as described in U.S. patent application Ser. No. 17/722,182 containing the complete pathway for retinoid synthesis. Corn mash waste obtained from a micro distillery was mixed with an artificial seawater medium at 6 and 20% corn mash by volume. In some embodiments, thin stillage from an ethanol plant is used in place of corn mash. The medium was supplemented with iron citrate. In some embodiments the corn mash can be used anywhere between 1% and 100% volume. In some embodiments, the corn mash is supplemented with glycerol. At high concentrations of corn mash, salts and minerals are added in either concentrated or solid form.

The medium was inoculated with a started culture at a dilution of 1:100 and grown with an overlay of a hydrophobic solvent such as dodecane, heptane, hexane, or vegetable oils for at least hours. The retinoids are extracted into the solvent layer and then can be separated via nanofiltration.

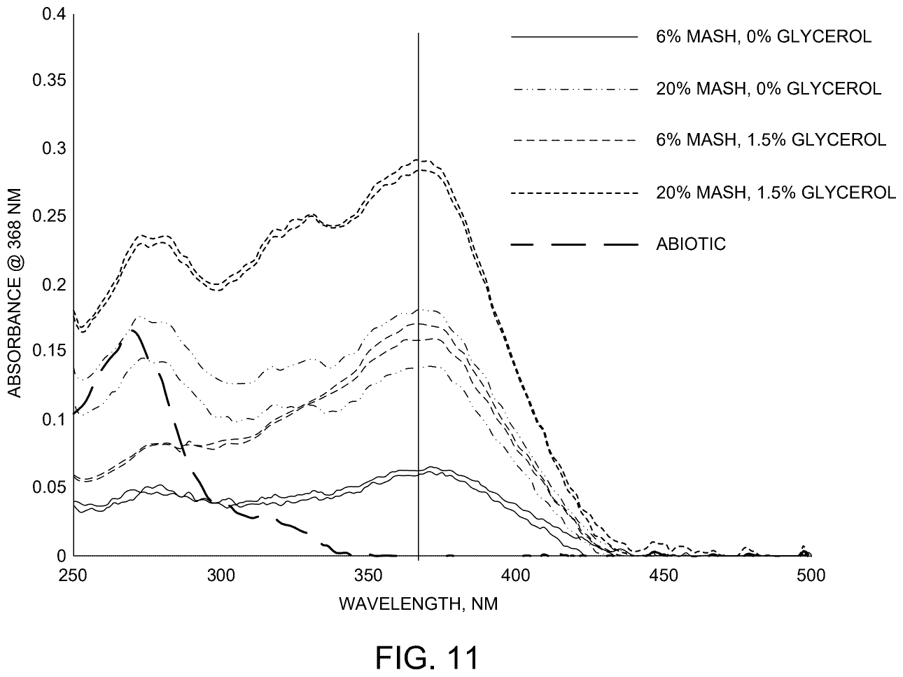

As indicated in the data shown in FIG. 11, retinoid production by the characteristic retinal absorbance peak at 368 retinoid production occurs across a range of concentrations of corn mash, both with and without supplemental glycerol. These data indicate that M. atlanticus is able to produce an industrially relevant quantity of retinoids from components of distillery/brewery waste and thin stillage.

While this invention has been particularly shown and described with references to preferred embodiments thereof, it will be understood by those skilled in the art that various changes in form and details may be made therein without departing from the scope of the invention encompassed by the appended claims.

1. A bioreactor system including at least one biofilm bioreactor containing an organism that can metabolize one or more components of a co-product stream and connections to the co-product stream to introduce said co-product stream into the bioreactor.

2. The bioreactor system of claim 1, wherein the co-product stream is from a corn ethanol plant.

3. The bioreactor system of claim 1, wherein the co-product stream is from a brewery or distillery.

4. The bioreactor system of claim 1, wherein the co-product stream is thin stillage.

5. The bioreactor system of claim 1, wherein the organism is M. atlanticus.

6. The bioreactor system of claim 1, wherein a pressure from the co-product stream provides the pressure to propel media through the bioreactor.

7. The bioreactor system of claim 1, further comprising a recirculating pump that recycles media through the at least one biofilm bioreactor and a valve between the co-product stream and recirculating line that allows for periodic introduction of material from the co-product stream.

8. The bioreactor system of claim 1, wherein the bioreactor system contains an emulsifier module to introduce corn oil into the reactor as the carbon source.

9. The bioreactor system of claim 1, further comprising a mixer for mixing salts with thin stillage prior to introduction into the at least one bioreactor.

10. The bioreactor system of claim 1, further comprising multiple bioreactors being run in parallel.

11. The bioreactor system of claim 1, wherein a product is retinol.

12. The bioreactor system of claim 1, wherein a product is retinaldehyde.

13. The bioreactor system of claim 1, wherein a product is retinoic acid.

14. A biofilm bioreactor system, comprising:
beads for holding a biofilm; and
oxygen permeable tubing to provide oxygen content during media recirculation.

15. A biofilm bioreactor containing oxygen permeable tubing where gas is piped through the reactor to improve oxygenation.

16. A method of using a biofilm bioreactor system to convert a co-product from a corn ethanol plant, a distillery or a brewery into a hydrophobic product.

17. The method of claim 16, where the hydrophobic product is retinol.

18. The method of claim 16, where the hydrophobic product is retinaldehyde.

19. The method of claim 16, where the hydrophobic product is retinoic acid.

20. A method of using a biofilm bioreactor system to convert thin stillage or distillers corn oil into a hydrophobic product.

21. A method of using a biofilm bioreactor system according to claim 1 to convert thin stillage or distillers corn oil into a hydrophobic product.

22. A bioreactor system comprising:
multiple biofilm bioreactors, each biofilm bioreactor containing an organism capable of metabolizing a co-product in a biorefinery to produce a hydrophobic chemical, each reactor being configured for circulating the co-product, for introducing at least one additional ingredient and/or for collecting the hydrophobic chemical;
sensors for assessing parameters in each of the biofilm bioreactor; and
a controller for controlling each of the biofilm bioreactors in response to an assessment of the sensors.

23. The bioreactor system of claim 22, wherein at least one additional ingredient is selected from a group consisting of a hydrophobic solvent, salts, nutrients and air.

24. The bioreactor system of claim 22, further comprising at least one pump for circulating the co-product.

25. The bioreactor system of claim 22, wherein each biofilm bioreactor is part of a loop for circulating the co-product.

26. The bioreactor system of claim 22, further comprising a feed valve for introducing the co-product.

27. The bioreactor system of claim 22, further comprising connections to connect the bioreactor system to a co-product stream.

28. The bioreactor system of claim 22, wherein the co-product is derived from a corn ethanol plant, a brewery or a distillery.

29. The bioreactor system of claim 22, wherein the biofilm bioreactors are arranged in parallel.

30. A corn ethanol plant, a brewery or a distillery comprising a co-product line connected to the bioreactor system of claim 22.

31. The corn ethanol plant of claim 30, wherein the co-product line is a thin stillage line or a distiller's corn oil line.

32. A method for preparing a hydrophobic chemical, the method comprising:
directing a co-product from a biorefinery to a bioreactor system that includes multiple biofilm bioreactors, each biofilm bioreactor containing an organism capable of metabolizing the co-product to produce the hydrophobic chemical;
circulating the co-product through at least one of the multiple biofilm bioreactors; and
operating at least one of the biofilm bioreactors in response to assessments from sensors associated with the at least one biofilm bioreactors.

33. The method of claim 32, further comprising introducing salts, nutrients and/or air to the at least one biofilm bioreactor.

34. The method of claim 32, further comprising introducing a hydrophobic solvent into the at least one biofilm bioreactor.

35. The method of claim 32, further comprising collecting the hydrophobic chemical.

36. The method of claim 32, further comprising separating the hydrophobic chemical from a hydrophobic solvent employed to extract the organic chemical from the at least one biofilm bioreactor.

37. A process for producing an alcohol in a biorefinery, comprising the method of claim 32.

38. The method of claim 32 wherein the co-product is derived from a line in a corn ethanol plant, a distillery or a brewery.
